(19) United States
(12) Patent Application Publication
(10) Pub. No.: US 2010/0304495 A1
(43) Pub. Date: Dec. 2, 2010

(54) Method for determining fruiting body and mycelium of Antrodia cinnamomea and Antrodia salmonea

(75) Inventors: Tun-Tschu CHANG, Taipei City (TW); Wu-Rong WANG, Taipei City (TW); Cheng-Jen Chou, Taipei City (TW)

Correspondence Address: Tun Tschu Chang, P.O. Box 46-563 Taipei, Taipei City, 10499, TW

(73) Assignee: TAIWAN FORESTRY RESEARCH INSTITUTE, Taipei City (TW)

(21) Appl. No.: 12/472,383

(22) Filed: May 27, 2009

(51) Int. Cl. G01N 33/50

(52) U.S. Cl. 436/129

(57) ABSTRACT

This invention relates to a method for determining fruiting body and mycelium of Antrodia cinnamomea and Antrodia salmonea. By using ten known components in Antrodia cinnamomea including five ergostanes (antcins C and K, zhankuic acids A, B and C), four lanostanes (sulphurenic acid, dehydrosulphurenic acid, eburicoic acid, dehydroeburicoic acid), and one monophenyl (4,7-dimethoxy-5-methyl-1,3-benzodioxole) as standards, differentiation of mycelia and basidiomes of Antrodia cinnamomea was carried out in the invention. The natural basidiomes collected from wood of Cinnamomum kanehirai at natural forests and the cultural basidiomes grown on potato dextrose agar medium contained all ten tested components. However, the natural mycelia collected from wood of C. kanehirai at a natural forest and the liquid/solid cultural mycelia grown on potato dextrose broth/potato dextrose agar media contained the four lanostanes and 4,7-dimethoxy-5-methyl-1,3-benzodioxole, but not the five ergostanes. These results indicate that the production of ergostanes is related to the basidiomatal formation of A. cinnamomea, but is not related to the grown substrates.

### BACKGROUND OF THE INVENTION

1. Technical Field

This invention relates to a method for determining fruiting body and mycelium of Antrodia cinnamomea and Antrodia salmonea.

2. Description of Related Art

Antrodia cinnamomea TT Chang & WN Chou is a resupinate to effused-reflexed basidiomycete with porous hymenium. It is known only from Taiwan and is restricted to Cinnamomum kanehirai Hayata. The basidiomes produced on the infested wood have been used as an herbal medicine in Taiwan. Owing to its host specificity and rarity in nature as well as effectiveness in curing certain illness, the basidiomes of the fungus are priced high. The artificial cultivation of A. cinnamomea basidiomes to satisfy market demand is considered the most effective solution. Researchers reported that A. cinnamomea produced basidiomes on artificial agar media without containing wood substrates of C. kanehirai indicating that the basidiomatal formation of A. cinnamomea was not related with the wood of C. kanehirai. In addition, other researchers reported that physical wounding of A. cinnamomea red hyphae induced basidiomatal formation on MEA plate.

It is said that wood substrates of C. kanehirai might be related with the bioactive components of A. cinnamomea. However, C. kanehirai is an endemic and endangered species in Taiwan. Resources of the wood are limited from natural forests. Therefore, it is impossible that C. kanehirai wood is used for cultivation of A. cinnamomea. It would be very interesting to know whether the basidiomes compose the bioactive components when they formed on the media without containing wood substrates of C. kanehirai. Methanolic/ethanolic extracts from natural basidiomes of A. cinnamomea had some bioactive components such as triterpenoids, monophenyl and biphenyl components. In addition, two lanostane-type triterpenoids were identified from cultural mycelia of A. cinnamomea and could be involved in the anti-inflammatory actions.

### BRIEF SUMMARY OF THE INVENTION

A method for determining fruiting body and mycelium of Antrodia cinnamomea and Antrodia salmonea is disclosed. The method comprising steps of:

providing a sample to be tested; and

separating and testing the sample so as to quantify a group of antcins and a group of zhankuic acids contained in the sample, wherein upon confirming the presence of one or more selected from the group consisting of antcin C, antcin K, zhankuic acid A, zhankuic acid B, and zhankuic acid C, the presence of fruiting body of Antrodia cinnamomea in the sample is determined.

### BRIEF DESCRIPTION OF THE SEVERAL VIEWS OF THE DRAWINGS

FIGS. 1 and 2 are HPLC chromatograms of the present invention.

### DETAILED DESCRIPTION OF THE INVENTION

Inventors of this invention compare the components from ethanolic extract of A. cinnamomea among natural basidiomes, cultural basidiomes, natural mycelia and liquid/agar cultural mycelia. Nine triterpenoids (antcins C, K, zhankuic acids A, B, C, sulphurenic acid dehydrosulphurenic acid, eburicoic acid and dehydroeburicoic acid) and one monophenyl component (4,7-dimethoxy-5-methyl-1,3-benzodioxole) from A. cinnamomea were purified and used as standards in analysis of high performance liquid chromatography (HPLC) to differentiation of mycelia and basidiomes of Antrodia cinnamomea in the invention.

### Materials and Methods

### Fungal Samples

Natural basidiomes: Three basidiomatal samples of A. cinnamomea were collected from the host C. kanehirai in natural forests at Hsinchu, Chiai and Kaohsiung Counties of Taiwan. Natural mycelia: The mycelial mats of A. cinnamomea grew in the decayed wood of C. kanehirai in natural forest at Hsinchu County were collected. Cultural basidiomes: Isolates TFRI B479 and B522 isolated from basidiomes of A. cinnamomea collected from Kaohsiung and Taoyuan Counties of Taiwan, respectively, were used for basidiomatal production on PDA (Bacto, potato-dextrose-agar) medium. One block (3×3 mm²) of culture was placed in the center of Petri dish containing PDA medium. Cultures were incubated at 24° C. in darkness for 3 months for basidiomatal formation. The basidiomes produced on the PDA medium were collected as cultural basidiomes. In addition, the mycelia near by the basidiomes were also collected for the HPLC analysis. Liquid cultural mycelia: Isolates TFRI B479 and B86 were used for liquid cultural mycelia. Four blocks (3×3 mm²) of cultures were inoculated into each 500 ml-flask containing 200 ml PDB (Himadia, potato-dextrose-broth) medium. Cultures were incubated at 24° C. in darkness for one month. At the end of the incubation, mycelia were rapidly washed with 500 ml of NaCl (250 mM) by an aspirator-suction system to remove contamination of the culture medium. Agar cultural mycelia: Isolates TFRI B479 and B86 were used for agar cultural mycelia. One block (3×3 mm²) of cultures was placed in the center of Petri dish containing PDA medium. Cultures were incubated at 24° C. in darkness for one month. At the end of the incubation, mycelial layer at the top of culture was removed from Petri dish. All test samples were dried with oven under 50° C. until dry before methanolic extracts.

### Preparation of Methanolic Extracts

Oven dry samples were refluxed four times with methanol for 6 h: The extracts were filtered and evaporated, then diluted with methanol to appropriated concentrations immediately before use.

### High Performance Liquid Chromatography (HPLC) System

HPLC was performed on an Agilent 1100 series with DAD detection. The detecting wavelength was set at 210 nm. Separations were obtained with a reversed phase column (Cosmosil 5C₁₈₋, AR-II, 250×4.6 mm, Kyoto, Japan) eluted at a flow rate of 1 ml min⁻¹ with a linear solvent gradient elution of A (0.0085% H₃PO₄ in H₂O), and B (acetonitrite) and the column was eluted according to the following profile: 0-65 min, 30-47% B, 65-100 min, 47-47% B, 110-140 min, 47-100% B, 140-170 min, 100-100% B, 170-175 min, 100-30% B, 175-200 min, 30-30% B. The column temperature was set at 30° C. The injection volume was 20 μL. Ten known compounds including five ergostane triterpenoids: antcins C, K, zhankuic acids A, B, and C, four lanostane triterpenoids: sulphurenic acid, dehydrosulphurenic acid, eburicoic acid, dehydroeburicoic acid, and one monophenyl: 4,7-dimethoxy-5-methyl-1,3-benzodioxole, were used as detected components. Each component and its retention times in parenthesis are followed: antcin K (23.1 and 24.3), 4,7-dimethethoxy-5-methyl-1,3-benzodioxole (35.8), antcin C (59.7), zhankuic acid C (62.6 and 64.0), zhankuic acid B (83.4 and 84.5), dehydrosulphurenic acid (83.5), sulphurenic acid (87.5), zhankuic acid A (93.9), dehydroeburicoic acid (140.1) and eburicoic acid (141.4).

### Results

### Comparison and Natural and Cultural Basidiomes

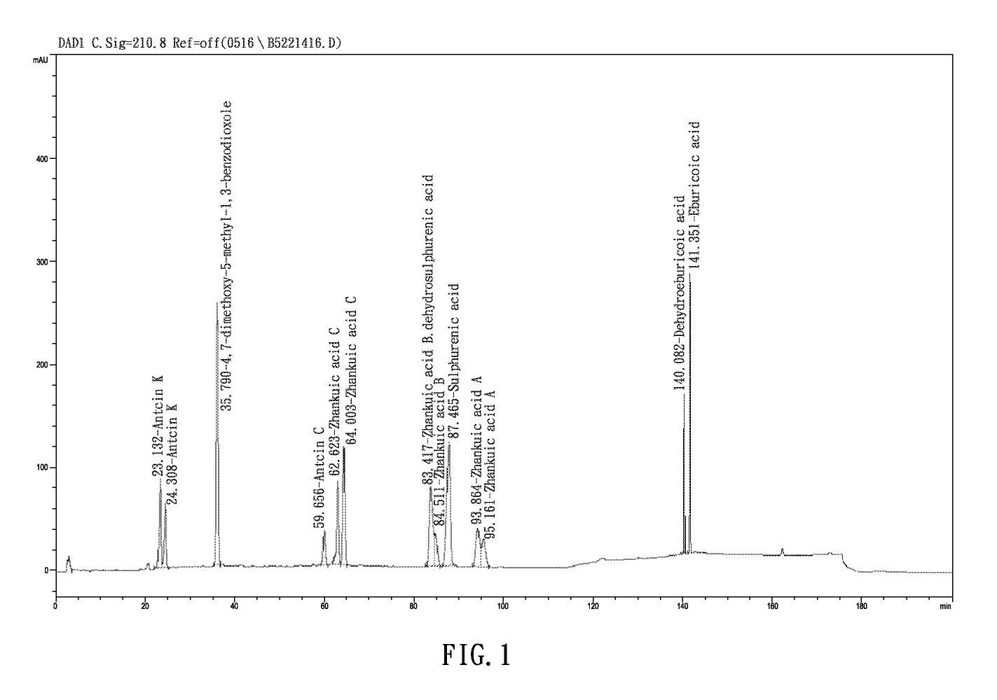

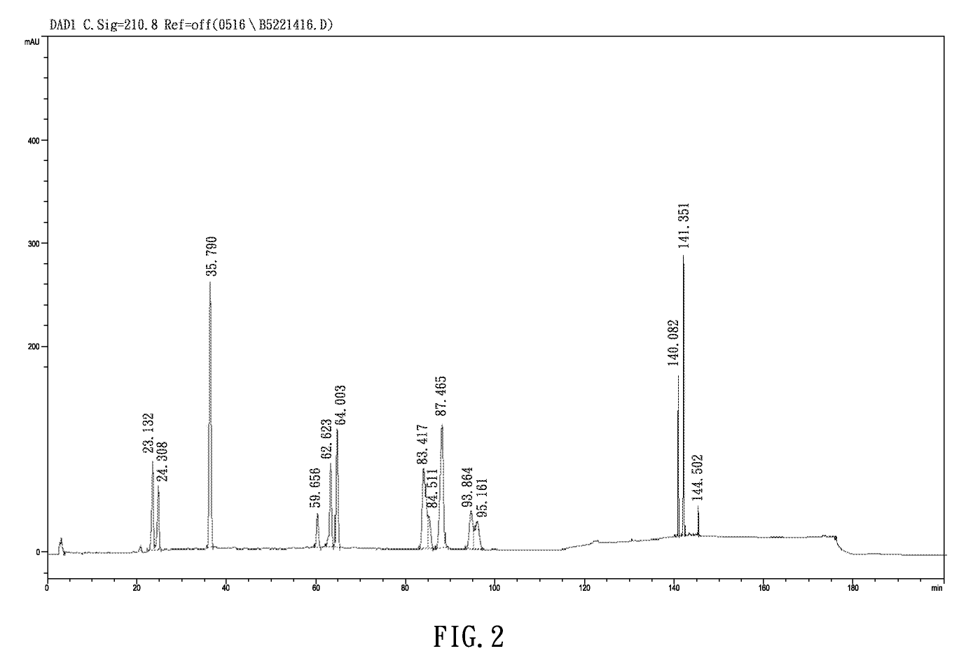

The HPLC (high performance liquid chromatography) chromatogram of ten tested components is presented in FIGS. 1 and 2 as standards. The HPLC chromatograms of three natural basidiomes from three collection sites (Hsinchu, Chiai and Kaohsiung Counties) were similar. Because they were similar, one chromatogram of a sample from Hsinchu County is present as standard of natural basidiomes. To compare with ten standard compounds (antcins C, K, zhankuic acids A, B, C, sulphurenic acid, dehydrosulphurenic acid, eburicoic acid, dehydroeburicoic acid and 4,7-dimethoxy-5-methyl-1,3-benzodioxole) by HPLC, these three natural basidiomes contained all the ten tested standard compounds. The HPLC chromatograms of two cultural basidiomes (isolates B479 and B522) were similar. To compare with ten standard compounds as natural basidiomes, these two cultural basidiomes also contained all the ten tested compounds.

### Comparison of Natural and Cultural Mycelia

The HPLC chromatograms (FIGS. 1 and 2) showed that the methanolic extracts of natural mycelia contained the four detected lanostane triterpenoids (sulphurenic acid, dehydrosulphurenic acid, eburicoic acid, dehydroeburicoic acid) and 4,7-dimethoxy-5-methyl-1,3-benzodioxole. The natural mycelia did not contain the five detected ergostane triterpenoids (antcins C, K, zhankuic acids A, B, C). The HPLC chromatograms of two liquid cultural mycelia and agar cultural mycelia (isolates B479 and B86) also contained the four lanostanes and one monophenyl compands as the natural mycelia. In addition, the mycelia near by the basidiomes from the nutrient agar medium of isolates TFRI B479 and B522 also only contained the four lanostanes and this monophenyl compound as the natural mycelia, but not the five detected ergostane triterpenoids produced by their cultural basidiomes in the same plates.

These results indicated the production of ergostane triterpenoids such as antcins C and K, zhankuic acids A, B and C are associated with basidiomatal formation of A. cinnamomea, because both natural basidiomes grown on wood of C. kanehirai and cultural basidiomes produced on PDA medium contained these five detected ergostane triterpenoids, but not in either natural or cultural mycelia (Table 1).

TABLE 1
Comparison of ten chemical components
between mycelia and basidiomes of Antrodia cinnamomea.

| Form of Antrodia | Chemical components^{a} | | | | | | | | | |
|---|---|---|---|---|---|---|---|---|---|---|
| cinnamomea | 1 | 2 | 3 | 4 | 5 | 6 | 7 | 8 | 9 | 10 |
| Basidiomes from nature | | | | | | | | | | |
| Hsinchu County | + | + | + | + | + | + | + | + | + | + |
| Chiai County | + | + | + | + | + | + | + | + | + | + |
| Kaohsiung County | + | + | + | + | + | + | + | + | + | + |
| Basidiomes from | | | | | | | | | | |
| culture | | | | | | | | | | |
| TFRI B479 | + | + | + | + | + | + | + | + | + | + |
| TFRI B522 | + | + | + | + | + | + | + | + | + | + |
| Mycelia from nature | − | − | − | − | − | + | + | + | + | + |
| Mycelia from culture | | | | | | | | | | |
| Liquid culture TFRI | − | − | − | − | − | + | + | + | + | + |
| B479 | | | | | | | | | | |
| TFRI B 86 | − | − | − | − | − | + | + | + | + | + |
| Agar culture TFRI B | − | − | − | − | − | + | + | + | + | + |
| 479 | | | | | | | | | | |
| TFRI B 86 | − | − | − | − | − | + | + | + | + | + |

^{a}1, antcin C; 2, antcin K; 3, zhankuic acid A; 4, zhankuic acid B; 5, zhankuic acid C; 6, sulphurenic acid; 7, dehydrosulphurenic acid; 8, eburicoic acid, 9, dehydroeburicoic acid; 10, 4,7-dimethoxy-5-methyl-1,3-benzodioxole.

The four detected lanostane triterpenoids (sulphurenic acid, dehydrosuphurenic acid, eburicoic acid and dehydroeburicoic acid) and one monophenyl (4,7-dimethoxy-5-methyl-1,3-benzodioxole) were produced by basidiomes and mycelia of A. cinnamomea regardless of the living substrates. Owing to prices of basidiomatal products of A. cinnamomea are quite higher than that of mycelia, it is said that some companies have used mycelia to substitute for basidiomes. It is almost impossible to identify the commercial products of A. cinnamomea are from mycelia or basidiomes when the products are dry powdered and capsulated/tableted. However, in this invention A. cinnamomea produced ergostane triterpenoids in its basidiomes, but not in it mycelia, so these results can be applied to the commercial quality check of this fungus.

Besides A. cinnamomea, basidiomes of A. salmonea, a close species of A. cinnamomea, also contained the five ergostane triterpenoids that have not been found in any other fungi. The monophenyl compound (4,7-dimethoxy-5-methyl-1,3-benzodioxole) was only found in A. cinnamomea indicated that it could be as an indicator component of A. cinnamomea. However, the four lanostane triterpenoids have been found in other fungi except dehydrosulphurenic acid that was only found in A. cinnamomea and A. salmomea. But, it is believed that dehydrosulphurenic acid should be presented in other fungi because its main structure is the same as sulphurenic acid.

In the invention, the production of the ten detected components is not related with the wood of C. kanehirai because they were presented in cultural mycelia and basidiomes grown on the substrated without containing wood of C. kanehirai. Five ergostanes, antcins C and K, and zhanknic acids A, B and C, isolated from basidiomes exhibited anti-inflammatory activity in isolated peripheral human neutrophils. An ergostane antcin C and three lanostanes (sulphurenic acid, eburicoic acid and dehydroeburicoic acid) isolated from basidiomes of A. cinnamomea contributed to the immuno-modulating activity. These bioactive compounds could be produced by cultural basidiomes and cultural mycelia grown on media without contained wood of C. kanehirai, indicating that artificial cultures of A. cinnamomea can bear some effective compounds.

Since the compounds of Antrodia salmonea are similar to that of Antrodia cinnamomea, the determining method can also be applied to Antrodia salmonea.

1. A method for determining the presence of fruiting body of Antrodia cinnamomea in a sample, the method comprising steps of:
providing a sample to be tested; and
separating and testing the sample so as to quantify a group of antcins and a group of zhankuic acids contained in the sample, wherein upon confirming the presence of one or more selected from the group consisting of antcin C, antcin K, zhankuic acid A, zhankuic acid B, and zhankuic acid C, the presence of fruiting body of Antrodia cinnamomea in the sample is determined.

2. The method of claim 1, wherein the group of antcins and the group of zhankuic acids in the sample are quantified by HPLC (High Performance Liquid Chromatography).

3. The method of claim 1, wherein the sample is pre-processed by being extracted with methanol.

4. The method of claim 1, wherein the sample is obtained from any one selected from the group consisting of medicines containing Antrodia cinnamomea components and compounds containing Antrodia cinnamomea components.

5. A method for determining the presence of fruiting body of Antrodia salmonea in a sample, the method comprising steps of:
providing a sample to be tested; and
separating and testing the sample so as to quantify a group of antcins and a group of zhankuic acids contained in the sample, wherein upon confirming the presence of one or more selected from the group consisting of antcin C, antcin K, zhankuic acid A, zhankuic acid B, and zhankuic acid C, the presence of fruiting body of Antrodia salmonea in the sample is determined.

6. The method of claim 5, wherein the group of antcins and the group of zhankuic acids in the sample are quantified by HPLC (High Performance Liquid Chromatography).

7. The method of claim 5, wherein the sample is pre-processed by being extracted with methanol.

8. The method of claim 5, wherein the sample is obtained from any one selected from the group consisting of medicines containing Antrodia salmonea components and compounds containing Antrodia salmonea components.
